(19) United States
(12) Patent Application Publication
(10) Pub. No.: US 2011/0092719 A1
(43) Pub. Date: Apr. 21, 2011

(54) Preparation of Escitalopram, Its Salts and Intermediates

(75) Inventors: Thota Giridhar, Hyderabad (IN); Gudipati Srinivasulu, Hyderabad (IN); Kotaru Srinivasa Rao, Hyderabad (IN)

(73) Assignee: SHODHANA LABORATORIES LIMITED, Hyderabad (IN)

(21) Appl. No.: 12/999,226

(22) PCT Filed: May 28, 2009
(86) PCT No.: PCT/IN2009/000309

§ 371 Date: Dec. 15, 2010

(30) Foreign Application Priority Data
Jun. 16, 2008 (IN) 1450/CHE/2008
Jan. 5, 2009 (IN) 17/CHE/2009

(51) Int. Cl. C07D 307/87; C07C 255/59; C07C 253/34

(52) U.S. Cl. 549/467; 558/422

(57) ABSTRACT

The present patent application relates to an improved process for the preparation of escitalopram, its salts and intermediates. It also relates to a novel crystalline form S of citalopram diol intermediate, process for preparation and its use in the preparation of citalopram, escitalopram and their salts.

### FIELD OF THE INVENTION

The present patent application relates to an improved process for the preparation of escitalopram, its salts and intermediates thereof. Further it relates to a novel crystalline form of citalopram diol intermediate, process for preparation and its use in preparing citalopram, escitalopram and its salts.

### BACKGROUND OF THE INVENTION

Citalopram is a well-known anti-depressant drug. It is chemically known as 1-[3-(dimethylamino) propyl]-1-(4-fluorophenyl)-1,3-dihydro-5-isobenzofurancarbonitrile and is described by the following structural Formula I.

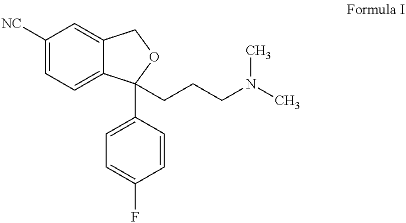

Escitalopram, which is the S-isomer of citalopram, is a selective, centrally acting serotonin reuptake inhibitor and is represented by Formula II. Pharmaceutical dosage forms containing the oxalate salt of escitalopram are sold in the market for oral administration.

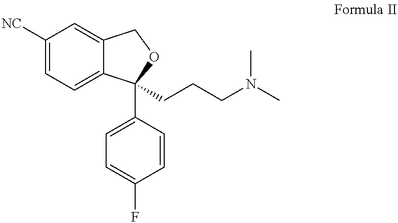

The citalopram diol as used in this application is represented by the structural Formula III and is chemically known as 4-(4-(dimethylamino)-1-(4-fluorophenyl)-1-hydroxybutyl)-3-(hydroxymethyl)benzonitrile.

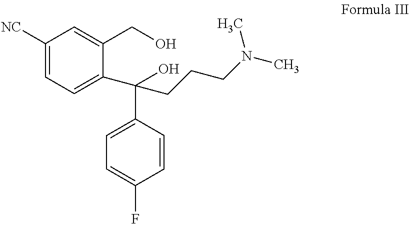

U.S. Pat. No. 4,650,884 discloses 4-(4-(dimethylamino)-1-(4-fluorophenyl)-1-hydroxybutyl)-3-(hydroxymethyl)benzonitrile, its salts as well as processes for their preparation and their use as intermediates in the preparation of citalopram.

U.S. Pat. No. 4,943,590 discloses Escitalopram, non-toxic acid addition salts thereof and processes for their preparation.

Processes for preparation of Escitalopram, its pharmaceutically acceptable salts and its intermediates have been described in: U.S. Pat. No. 6,762,307; International Application Publication Nos. WO 2006/106531, WO 2006/136169, and WO 2006/025071; U.S. Patent Application Publication Nos. 2006/0009515.

Many of the processes reported for the preparation of citalopram diol involves recovery citalopram diol either in the form of residue, which is contaminated with impurities or in the form of solid HBr salt, which when used in the subsequent step results in lower yield.

U.S. Pat. No. 7,435,838 B2 discloses crystalline forms of citalopram diol having two different differential scanning calorimetry (DSC) patterns. Citalopram diol when crystallized from mixture of isopropyl ether and n-heptane was found to have melting point 98.63° C.; the peak value is 104.18° C. and when crystallized from mixture of ethanol and water was found to have melting point 51.69° C.; the peak value is 59.28° C. The mixture of solvents used in the '838 patent are either hazardous to use in commercial manufacturing or difficult to recover and reuse.

Therefore there is a need for a process, which is advantageous in preventing racemization, so as to increase the yields of the final product and also to yield an enantiomerically pure form of escitalopram and its pharmaceutically acceptable salts.

### SUMMARY OF THE INVENTION

In first aspect, the present patent application relates to a process for preparation of escitalopram or a salt thereof comprising:

a) reacting 5-Cyano phthalide with 1-(4-fluorophenyl)magnesium halide and 1-[3-(dimethylamino) propyl]magnesium halide to obtain citalopram diol of Formula III;

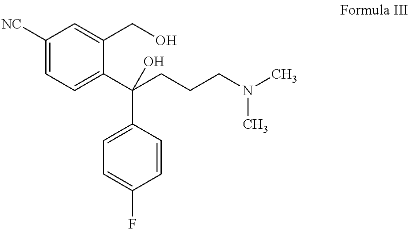

b) reacting the citalopram diol of Formula III with an optically pure di-para-toluoyl tartaric acid (DPTTA) to obtain (−)4-(4-(dimethylamino)-1-(4-fluorophenyl)-1-hydroxybutyl)-3-(hydroxymethyl)benzonitrile (+)DPTTA salt of Formula IV in solid form;

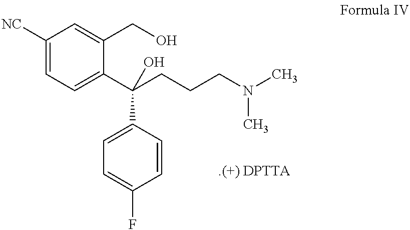

c) converting the (+)DPTTA salt of 4-(4-(dimethylamino)-1-(4-fluorophenyl)-1-hydroxybutyl)-3-(hydroxymethyl)benzonitrile (Formula IV) to escitalopram of Formula II or a salt thereof.

In second aspect, the present application provides a process for the preparation of pure escitalopram oxalate comprising:

a) providing a mixture of escitalopram oxalate and an alcohol;

b) removing the un-dissolved solid; and

c) recovering the pure escitalopram oxalate from the mother liquors.

In third aspect, the present application provides a novel crystalline form (designated as ‘Form S″) of citalopram diol, characterized by having an endotherm at 77.7±3° C. as measured by DSC.

In fourth aspect, the present application relates to a process for the preparation of crystalline Form S of citalopram diol comprising crystallizing citalopram diol from a solvent medium comprising an aromatic hydrocarbon.

In fifth aspect, the present application provides a process comprising converting crystalline Form S of citalopram diol intermediate to citalopram escitalopram and their salts.

In the final aspect the present application relates to a process for the preparation of citalopram or a salt thereof comprising reacting citalopram diol intermediate with p-toluenesulfonyl chloride.

### BRIEF DESCRIPTION OF THE DRAWINGS

FIG. 1 is an illustrative DSC thermogram of citalopram diol crystalline Form S obtained in Example 4.

FIG. 2 is an illustrative X-ray powder diffraction pattern of citalopram diol crystalline Form S obtained in Example 4.

### DETAILED DESCRIPTION OF THE INVENTION

In first aspect, the present patent application relates to a process for preparation of escitalopram or a salt thereof comprising:

a) reacting 5-Cyano phthalide with 1-(4-fluorophenyl)magnesium halide and 1-[3-(dimethylamino) propyl]magnesium halide to obtain citalopram diol of Formula III;

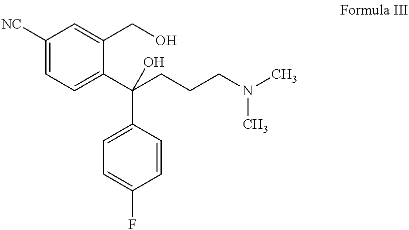

b) reacting the citalopram diol of Formula III with an optically pure di-para-toluoyl tartaric acid (DPTTA) to obtain (−)4-(4-(dimethylamino)-1-(4-fluorophenyl)-1-hydroxybutyl)-3-(hydroxymethyl)benzonitrile (+)DPTTA salt of Formula IV in solid form;

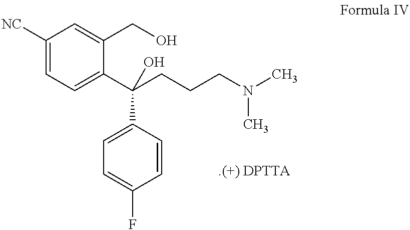

c) converting the (+)DPTTA salt of (−)₄-(4-(dimethylamino)-1-(4-fluorophenyl)-1-hydroxybutyl)-3-(hydroxymethyl)benzonitrile (Formula IV) into escitalopram of Formula II or a salt thereof.

In step a) 1-(4-fluorophenyl)magnesium halide that may be used include 1-(4-fluorophenyl)magnesium chloride, 1-(4-fluorophenyl) magnesium bromide or 1-(4-fluorophenyl)magnesium iodide.

1-[3-(dimethylamino) propyl]magnesium halide that may be used include 1-[3-(dimethylamino) propyl]magnesium chloride, 1-[3-(dimethylamino) propyl]magnesium bromide, 1-[3-(dimethylamino) propyl]magnesium iodide.

1-(4-fluorophenyl)magnesium halide and 1-[3-(dimethylamino) propyl]magnesium halide (Grignard reagents) that may be used in the process of step a) include suitable commercially available reagents, which are stored in an inert atmosphere or they may be freshly prepared just before the reaction.

Solvents which may be used in the process of Step a) include and are not limited to ethers such as for example tetrahydrofuran (THF), 2-methyl THF, diethyl ether, diisopropyl ether, methyl tertiary-butyl ether, petroleum ether; and the like; and mixtures thereof.

A suitable temperature for conducting the reaction may range from about −20° C. to about 100° C. or from about 0° C. to about 15° C.

The molar ratios of both 1-(4-fluorophenyl) halide and 1-[3-(dimethylamino) propyl]halide used in the reaction are optimized in establishing the cost of the process, since these are very expensive raw materials. Only a sufficient amount of raw material is used so that it is utilized completely in the reaction. An excess molar amount of 1-(4-fluorophenyl) halide and 1-[3-(dimethylamino) propyl]halide not only have impact on the purity of the product, but also the cost would be increased.

The molar ratio of 1-(4-fluorophenyl) halide may range from about 1.2 to about 1.5, preferably about 1.3 to 1.4, per mole of 5-Cyano phthalide.

The molar ratio of 1-[3-(dimethylamino) propyl]halide with respect to 5-Cyano phthalide may range from about 1.2 to about 1.5, preferably about 1.4 to 1.5.

After completion, the reaction mixture may be quenched with cold water and acidified by adding mineral acid such as for example hydrochloric acid.

The reaction product may then be recovered by extraction of the reaction mixture with a suitable organic solvent. The organic solvent that may be used for extraction of the product include and are not limited to halogenated solvents such as for example dichloromethane, dichloroethane and chloroform; hydrocarbon solvents such as for example n-hexane, n-heptane, toluene, xylene and the like; ester solvents such as for example ethyl acetate, butyl acetate; ether solvents such as for example diisopropyl ether, dibutyl ether and mixtures thereof.

The organic layer containing the product may be used in the next step directly or it can be distilled to obtain the product as residue.

The residue obtained may be used in the next step directly by dissolving in a suitable solvent or it can be triturated with a suitable solvent to recover the product in solid form, preferably in crystalline from.

It is advantageous to use the citalopram diol directly to react with optically pure DPTTA, without converting to an acid addition salt such as HBr, which leads to increase in process time, effluent and reduction in yield.

Step b) involves reacting the citalopram diol of Formula III with an optically pure di-para-toluoyl tartaric acid (DPTTA) to obtain (−)4-(4-(dimethylamino)-1-(4-fluorophenyl)-1-hydroxybutyl)-3-(hydroxymethyl) benzonitrile (+)DPTTA salt of Formula IV in solid form;

Citalopram diol intermediate that is used in the process of step b) may be in the solid form or in the form a liquid or it may be in the form of an organic layer obtained from the previous processing step.

DPTTA that may be used include optically pure form such as (+) DPTTA or (−)DPTTA.

The citalopram diol intermediate may be reacted with (+)DPTTA to form S-citalopram diol (+)DPTTA salt as solid. Alternatively the citalopram diol intermediate may be reacted with (−)DPTTA to remove the R-citalopram diol (−)DPTTA salt as solid and reacting the non-racemic citalopram diol obtained from the mother liquors with (+)DPTTA to form S-citalopram diol (+) DPTTA salt as solid as per the procedure described in our co-pending PCT application no. PCT/IN2009/000092.

Solvents that may be used in step b) include and are not limited to alcohols such as for example methanol, isopropanol, ethanol and the like; ketones such as for example acetone, ethyl methyl ketone and the like; ester solvents such as for example ethyl acetate, butyl acetate; ether solvents such as for example diisopropyl ether, dibutyl ether and mixtures thereof.

The solid product is recovered by suitable techniques such as decantation, filtration by gravity or by suction, centrifugation, and the like. Other techniques for separating the solids from the reaction mixtures are also within the scope of this invention.

Step c) involves converting the (+)DPTTA salt of (−)4-(4-(dimethylamino)-1-(4-fluorophenyl)-1-hydroxybutyl)-3-(hydroxymethyl) benzonitrile (Formula IV) into escitalopram of Formula II or a salt thereof.

The (−)4-(4-(dimethylamino)-1-(4-fluorophenyl)-1-hydroxybutyl)-3-(hydroxymethyl) benzonitrile(+)DPTTA salt of Formula IV is suitably converted into its freebase form by treating with a suitable base.

(−)4-(4-(dimethylamino)-1-(4-fluorophenyl)-1-hydroxybutyl)-3-(hydroxymethyl) benzonitrile free base is then reacted with methane sulfonyl chloride or p-toluene sulfonyl chloride in the presence of a suitable organic base.

Organic base that may be used include and are not limited to triethylamine, diisopropyl amine, diisopropyl ethyl amine and the like.

Suitably methane sulfonyl chloride or p-toluene sulfonyl chloride may be added slowly to the reaction mixture.

Organic solvents that may be used for reaction include and are not limited to halogenated solvents such as for example dichloromethane; hydrocarbon solvents such as for example n-hexane, n-heptane, toluene, xylene and the like; and mixtures thereof.

After completion, the reaction mixture may be quenched with water and the product may then be recovered by extraction of the reaction mixture with a suitable organic solvent. Organic solvents that may be used for extraction of the product include and are not limited to halogenated solvents such as for example dichloromethane, chloroform; hydrocarbon solvents such as for example n-hexane, n-heptane, toluene, xylene and the like; ester solvents such as for example ethyl acetate, butyl acetate; and mixtures thereof.

The organic layer containing the product may be used in the next step directly or it may be distilled to obtain the product as residue.

Escitalopram free base thus obtained may be converted into a desired pharmaceutically acceptable acid addition salt using conventional methods by reacting with a pharmaceutically acceptable acid.

Pharmaceutically acceptable acids that can be used for preparing the salt of escitalopram include and are not limited to: inorganic acids such as for example hydrochloric acid, hydrobromic acid; and organic acids such as for example acetic acid, tartaric acid, oxalic acid, methanesulfonic acid and the like, preferably oxalic acid.

In second aspect the present application provides a process for the preparation of pure escitalopram oxalate comprising:

a) providing a mixture of escitalopram oxalate and an alcohol;

b) removing the un-dissolved solid; and

c) recovering the pure escitalopram oxalate from the mother liquors.

The step of providing a mixture of escitalopram oxalate and an alcohol include mixing escitalopram oxalate with an alcohol solvent or the mixture may be obtained from a previous processing step where escitalopram oxalate is prepared in an alcohol solvent.

In one embodiment, the providing step includes mixing solid escitalopram oxalate in an alcohol solvent. The starting escitalopram oxalate may be of any form such as crystalline, amorphous or mixture of crystalline and amorphous forms.

The concentration of escitalopram oxalate in the mixture may generally range from about 0.5 gm/ml to about 1 gm/ml in the solvent.

The mixing may be carried out at a suitable temperature such as 10-50° C., preferably 20-40° C. However one skilled in the art may choose the suitable temperature depending on the concentration of the escitalopram oxalate in the given solvent, which is within the scope of the present invention.

In another embodiment, the providing step includes dissolving free base of escitalopram in an alcohol solvent, treating the free base solution with oxalic acid to obtain escitalopram oxalate in-situ.

The alcohol solvents include C1-C4 alcohols and mixtures thereof with water. The particular solvents suitable for the providing step include methanol, ethanol, n-propanol, isopropanol, n-butanol, isobutanol, t-butanol, water and their mixtures, preferably methanol.

Step b) involves removing the un-dissolved solid by using conventional filtration methods such as filtration by gravity or by suction, centrifugation and the like.

The un-dissolved solid obtained during the filtration is escitalopram oxalate with enriched R-isomer content. Without bound by particular theory, it is believed that the R-isomer content is enriched in the un-dissolved solid due to variation in its solubility at the said temperature and concentration in the said solvent.

Step c) involves recovering the pure escitalopram oxalate from the mother liquors.

The mother liquors containing pure escitalopram oxalate may treated with activated charcoal at about 25-50° C.

The mother liquor is suitably distilled to remove the solvent present in it to obtain a residue, which may then be dissolved in another suitable solvent. Solvent may be removed by distillation with or without vacuum at elevated temperatures such as about 20° C. to about 70° C. Any temperature and vacuum conditions can be used as long as there is no increase in the impurity levels of the product.

For re-crystallization, a solution can be prepared at an elevated temperature if desired to achieve a desired concentration. Any temperature is acceptable for the dissolution as long as a clear solution of the escitalopram oxalate is obtained and is not detrimental to the drug substance chemically or physically. The exact temperature required can be readily determined by a person skilled in the art and will also depend on parameters such as concentration. The solution may be brought down to room temperature for further processing if required otherwise; an elevated temperature may be used.

For isolation to occur, the reaction mass may be maintained further at temperatures lower than the concentration temperatures such as for example below about 10° C. to about 25° C., for a period of time as required for a more complete isolation of the product. The exact cooling temperature and time required for complete isolation can be readily determined by a person skilled in the art and will also depend on parameters such as concentration and temperature of the solution or slurry. Optionally, isolation may be enhanced by methods such as cooling, partial removal of the solvent from the mixture, by adding an anti-solvent to the reaction mixture, or a combination thereof.

The solid material isolated is recovered from the final mixture, with or without cooling below the operating temperature, using techniques such as filtration by gravity, or by suction, centrifugation, and the like. The crystals so isolated will carry a small proportion of occluded mother liquor containing a higher percentage of impurities. If desired the crystals can be washed with a solvent to wash out the mother liquor. Optionally, the solid isolated may be further dried. Drying can be carried out at reduced pressures at temperatures such as about 35° C. to about 70° C. The drying can be carried out for any desired time period that achieves a desired purity, for example, about 1 to 10 hours, or longer. Drying may also be carried out for shorter or longer periods of time depending on the product specifications. The exact time required can be readily determined by a person skilled in the art.

By the term “pure escitalopram oxalate”, it is meant that escitalopram oxalate prepared in accordance with the present invention contains less than about 1.0%, or less than about 0.5%, by weight of the corresponding impurities like the R-isomer of Citalopram, as characterized by a high performance liquid chromatography (“HPLC”) chromatogram obtained from a mixture comprising the desired compound and the said impurity.

“R-isomer of Citalopram” refers to of Formula IIa;

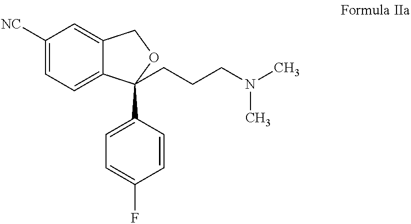

Escitalopram oxalate having a reduced level of impurities typically also contains residual solvents. For purposes of the present invention, any residual solvents in pure escitalopram oxalate are present at very low concentrations. Residual solvents can be quantified by application of chromatographic techniques, such as gas chromatography.

In another embodiment, there is provided a process for the preparation of pure escitalopram oxalate comprising:

a) providing a mixture of escitalopram oxalate and aqueous acetone;

b) recovering the pure escitalopram oxalate.

The step of providing a mixture of escitalopram oxalate and aqueous acetone include mixing escitalopram oxalate with aqueous acetone or the mixture may be obtained from a previous processing step where escitalopram oxalate is prepared in aqueous acetone solvent.

In one embodiment, the providing step includes mixing solid escitalopram oxalate in aqueous acetone. The starting escitalopram oxalate may be of any form such as crystalline, amorphous or mixture of crystalline and amorphous forms.

The concentration of escitalopram oxalate in the mixture may generally range from about 0.5 gm/ml to about 1 gm/ml in the solvent.

The mixing may be carried out at a suitable temperature such as 30-60° C., preferably 40-50° C.

In another embodiment, the providing step includes dissolving free base of escitalopram in aqueous acetone, treating the free base solution with oxalic acid to obtain escitalopram oxalate in-situ.

Aqueous acetone as used here in may contain water from about 1 to about 10% w/w.

In step b) the reaction mass may be maintained further at temperatures lower than the mixing temperatures such as for example 25-30° C., for a period of time as required for a more complete isolation of the product.

The solid material isolated is recovered from the final mixture, with or without cooling below the operating temperature, using techniques such as filtration by gravity, or by suction, centrifugation, and the like. The crystals so isolated will carry a small proportion of occluded mother liquor containing a higher percentage of impurities. If desired the crystals can be washed with a solvent to wash out the mother liquor. Optionally, the solid isolated may be further dried. Drying can be carried out at reduced pressures at temperatures such as about 35° C. to about 70° C. The drying can be carried out for any desired time period that achieves a desired purity, for example, about 1 to 10 hours, or longer. Drying may also be carried out for shorter or longer periods of time depending on the product specifications. The exact time required can be readily determined by a person skilled in the art.

The pure escitalopram oxalate obtained by the afore said process is having single maximum impurity less than 0.1% w/w as determined by HPLC.

In third aspect the present application provides a novel crystalline form (designated as ‘Form S”) of citalopram diol, characterized by having an endotherm at 77.7±3° C. as measured by DSC.

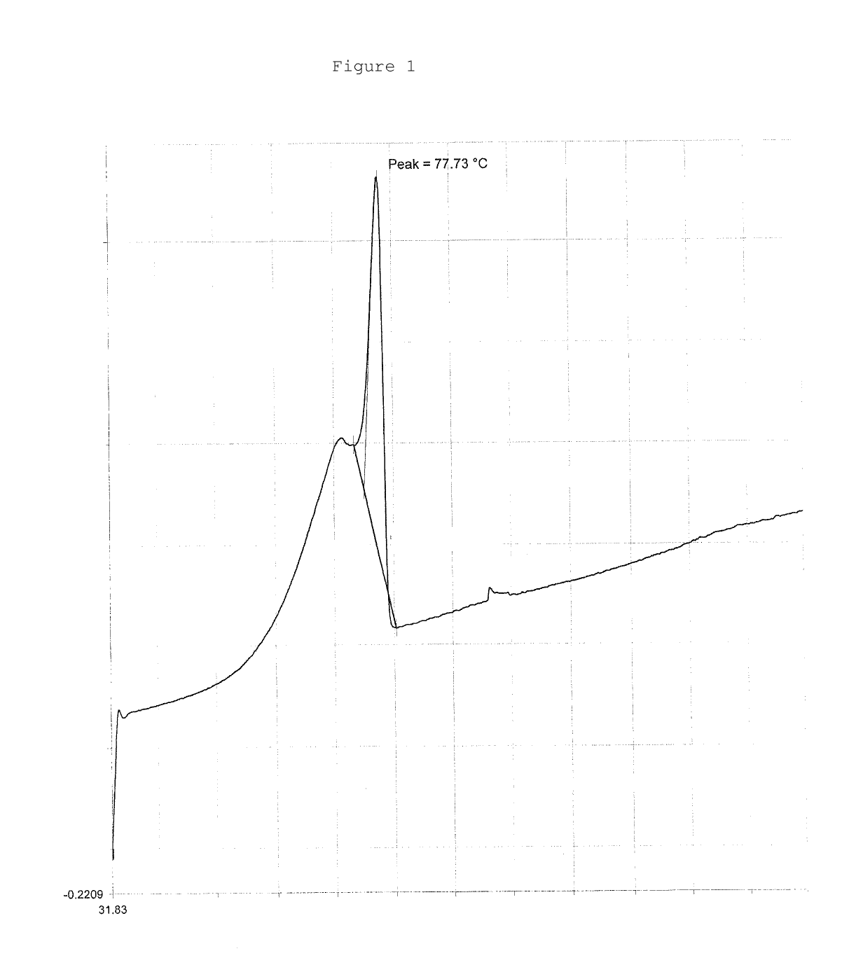

Citalopram diol crystalline Form S of the present application is further characterized by having DSC thermogram pattern substantially as shown in FIG. 1.

Differential scanning calorimetric analysis was carried out on TAQ1000 and the thermogram was recorded from 30° C. to 150° C. under the nitrogen flow of 50 mL/min at a heating rate of 2° C./min.

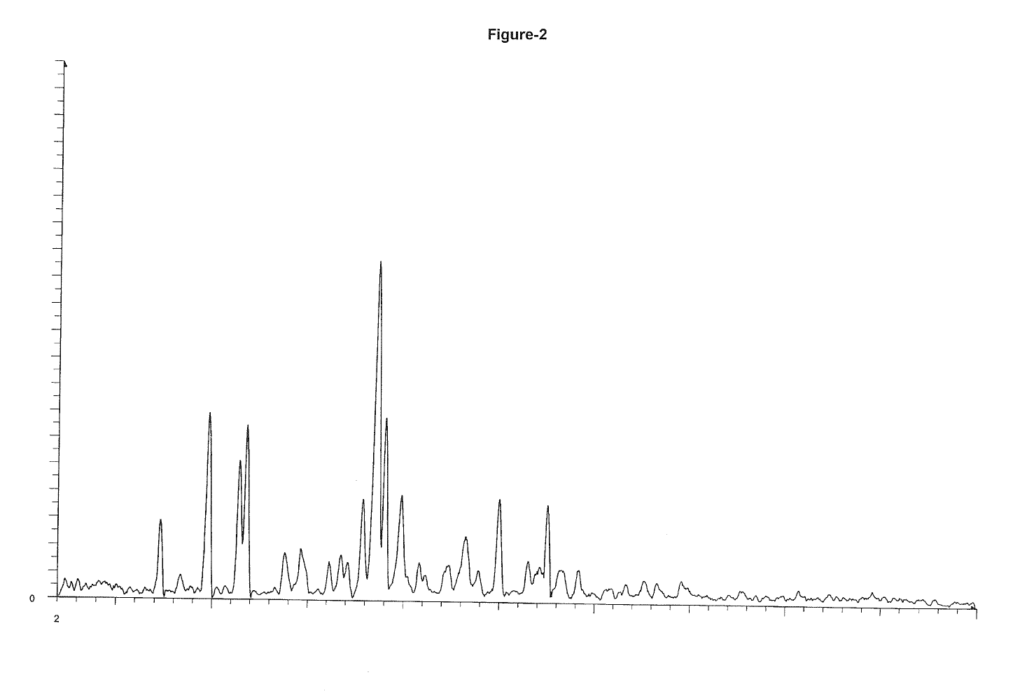

Citalopram diol crystalline Form S of the present application is having an X-ray powder diffraction pattern, as shown in FIG. 2, comprising peaks expressed in degrees 2θ that are selected from 7.36±0.2, 8.43±0.2, 9.77±0.2, 11.79±0.2, 16.86±0.2, 20.08±0.2 and 23.27±0.2.

In fourth aspect the present application relates to a process for the preparation of crystalline Form S of citalopram diol comprising crystallizing citalopram diol from a solvent medium comprising an aromatic hydrocarbon.

The citalopram diol intermediate is combined with the solvent or such a solution may be obtained from a previous processing step. The citalopram diol intermediate that is used as input material in the present application may be in any form such as in the form of oil, amorphous form or any crystalline form other than Form S. It may also be obtained in-situ by any process known in the art including the process described in the present application.

Suitable aromatic hydrocarbon solvent that may be used for crystallization of the product include and are not limited to toluene, xylene and the like; preferably toluene.

The citalopram diol intermediate solution may have concentration ranging from about 0.2 gm/ml to about 1 gm/ml in the solvent.

For isolation to occur, the reaction mass may be maintained further at temperatures of about 10° C. to about 25° C., for a period of time as required for complete isolation of the product. The exact cooling temperature and time required for complete isolation can be readily determined by a person skilled in the art and will also depend on parameters such as concentration and temperature of the solution or slurry. Optionally, isolation may be enhanced by methods such as cooling, partial removal of the solvent from the mixture, by adding an anti-solvent to the reaction mixture, or a combination thereof.

The solid material isolated is recovered from the final mixture, with or without cooling below the operating temperature, using techniques such as filtration by gravity, or by suction, centrifugation, and the like. The crystals so isolated will carry a small proportion of occluded mother liquor containing a higher percentage of impurities. If desired the crystals can be washed with a solvent to wash out the mother liquor. Optionally, the solid isolated may be further dried. Drying can be carried out at reduced pressures at temperatures such as about 25° C. to about 40° C. The drying can be carried out for any desired time period that achieves a desired purity, for example, about 1 to 10 hours, or longer.

The crystalline Form S of citalopram diol intermediate has purity greater than 99% or preferably greater than 99.5% w/w as determined by HPLC. It has commercially sufficient chemical and polymorphic stability on long-term storage and can be used in the manufacture of citalopram, escitalopram or salts thereof.

The organic solvents used for the crystallization of Form S of citalopram diol intermediate are relatively safe on commercial scale and easy to recover and reuse.

In fifth aspect the present application provides a process comprising converting crystalline Form S of citalopram diol intermediate to citalopram, escitalopram and their salts.

The crystalline Form S of citalopram diol intermediate may be used as starting material for the preparation of highly pure citalopram and its acid addition salts by any process known in the art including the one described in the present application.

The acid addition salts that includes but are not limited to hydrochloric acid, hydrobromic acid, oxalic acid, preferably hydrobromide salt of citalopram.

The process for the preparation of escitalopram of Formula II or a salt thereof comprising

a) resolution of crystalline Form S of citalopram diol intermediate to obtain optically pure S-citalopram diol intermediate; and

b) converting the optically pure S-citalopram diol intermediate in to escitalopram or salts thereof.

The resolution of citalopram diol intermediate can be carried out by treating the crystalline Form S of citalopram diol intermediate with an optically pure acid such as Di-p-toluoyl tartaric acid (DPTTA) in presence of a suitable solvent.

DPTTA that may be used include optically pure form such as (+) DPTTA

Suitable solvent that may be used include but are not limited to alcohols such as methanol, ethanol, isopropanol and the like. Ketones such as acetone, ethyl methyl ketone and the like or mixtures thereof.

The acid addition salts that includes but are not limited to hydrochloric acid, hydrobromic acid, oxalic acid, preferably oxalic acid salt of S— citalopram

In the final aspect the present application relates to process for the preparation of citalopram or a salt thereof comprising reacting citalopram diol intermediate with p-toluenesulfonyl chloride.

The citalopram diol intermediate that is used to react with p-toluenesulfonyl chloride may be in the form of non-crystalline form or crystalline form including crystalline form S of the present application.

Suitable solvents that may be used includes, but are not limited to aromatic hydrocarbons such as toluene, xylene and the like; alkyl ester solvent such as ethyl acetate, propyl acetate and the like or mixtures thereof.

Suitably the reaction may be carried out at temperature from about 15° C. to about 30° C. and for about 30 minutes to about 3 hours.

It is advantageous to use p-toluenesulfonyl chloride for ring closure as the reaction proceeds under mild conditions resulting in formation of pure product.

After completion, the reaction mixture may be quenched with water, organic layer containing the product is separated and citalopram is recovered by conventional methods.

In one embodiment the organic layer is treated with activated charcoal, distilled off completely to obtain residue and crystallized from methanol.

Citalopram that is obtained by the above process is having purity more than 99% w/w, preferably more than 99.5% w/w as determined by HPLC.

Pure citalopram obtained by the above process is converted to acid addition salt that includes hydrochloric acid, hydrobromic acid, oxalic acid, preferably hydrobromide salt of citalopram.

Certain specific aspects and embodiments of the present application will be explained in more detail with reference to the following examples, which are provided by way of illustration only and should not be construed as limiting the scope of the invention in any manner

### EXAMPLES

### Example 1

### Preparation of (−)4-(4-(dimethylamino)-1-(4-fluorophenyl)-1-hydroxybutyl)-3-(hydroxymethyl)benzonitrile (+)DPTTA salt

### a) Preparation of 1-(4-fluorophenyl)magnesium bromide

10 gm of Mg turnings was charged in a flask containing 110 ml of dry THF and heated to about 80° C. A pinch of molecular Iodine was added to the reaction contents. A mixture of THF (110 ml) and P-fluoro bromobenzene (72 gm) was added slowly to the reaction mixture at 80-85° C. for about 2 hours. The reaction mixture was stirred at reflux for about 3 hours and the whole reaction mixture was used directly in the reaction.

### b) Preparation of 1[3-(dimethylamino) propyl]magnesium chloride

56 gm of KOH flakes were added in a flask containing water (170 ml) and stirred for clear solution. The solution was cooled to 0-5° C. and 156 gm of 1-[3-(dimethylamino) propyl]chloride HCl salt was charged. Separated the upper layer containing the 1-[3-(dimethylamino) propyl]chloride and kept aside. Aqueous layer was extracted with toluene (3×50 ml) and the organic layer was mixed with the above 1-[3-(dimethylamino) propyl]chloride layer separated initially. Dry the total organic layer with KOH till the moisture content is below 0.1% w/w.

12 gm of Mg turnings was charged in a flask containing 50 ml of dry THF and heated to about 100° C. A pinch of molecular Iodine was added to the reaction contents. A mixture of THF (120 ml) and organic layer containing 1-[3-(dimethylamino) propyl]chloride obtained above was added slowly to the reaction mixture at 100-110° C. for about 2 hours. The reaction mixture was stirred at reflux for about 2 hours and the whole reaction mixture was used directly in the reaction.

### c) Preparation of 4-(4-(dimethylamino)-1-(4-fluorophenyl)-1-hydroxybutyl)-3-(hydroxymethyl)benzonitrile

50 gm of 5-Cyano phthalide was stirred in 150 ml of THF at −5° C. 227 gm of 1-(4-fluorophenyl)magnesium bromide solution obtained in above step a) was added to the above solution and stirred for about 15 minutes at −5° C. 324 gm of 1-[3-(dimethylamino) propyl]magnesium chloride solution obtained in step b) was added slowly to the reaction mass at −5° C. and then stirred for about 30 minutes at room temperature. The reaction mixture was quenched into cold water (650 ml). Reaction mass pH was adjusted to about 2.0 with 36% aqueous hydrochloric acid (94 ml) and washed the reaction mixture with toluene (2×150 ml). The aqueous layer was separated, toluene (300 ml) was added and pH was adjusted to about 7.5 with aqueous ammonia (45 ml). The organic layer was separated and aqueous layer was extracted with toluene (1×200 ml; 1×100 ml). Total organic layer was washed with water (1×250 ml) followed by saturated aqueous sodium chloride solution (1×250 ml). The final organic layer was distilled completely under vacuum to get the residue of 70 gm.

### d) Preparation of (−)4-(4-(dimethylamino)-1-(4-fluorophenyl)-1-hydroxybutyl)-3-(hydroxymethyl)benzonitrile (+)DPTTA salt

350 ml of isopropyl alcohol was charged to the residue and heated and stirred, then cooled to room temperature. 35 gm of (+)-DPTTA was added to the above solution and stirred for one hour at room temperature to isolation of the solid. The reaction mixture was heated to about 40-45° C., stirred for about 2 hours. The reaction mixture was cooled to room temperature, filtered the solid and dried to get 39 gm of the title compound.

### Example 2

### Preparation of Escitalopram Oxalate

25 gm of (−)-4-(4-(dimethylamino)-1-(4-fluorophenyl)-1-hydroxybutyl)-3-(hydroxymethyl)benzonitrile (+)-DPTTA salt was stirred in water; pH was adjusted to about 9.0 with aqueous ammonia solution. The reaction mixture was extracted with toluene (1×200 ml, 2×100 ml) and the total organic layer was washed with water. The organic layer was further washed with saturated aqueous sodium chloride solution and final organic layer was distilled off completely to obtain 15 gm of residue.

The residue was charged in 150 ml dichloromethane and stirred. 33.5 ml of triethylamine was charged and the reaction mixture was cooled to −5° C. A mixture of dichloromethane (14 ml) and methane sulfonyl chloride (4.5 ml) was added slowly. The reaction mixture was maintained for about 3 hours. After completion, the reaction mixture was washed with aqueous saturated sodium chloride solution (100 ml). The organic layer was distilled completely and 150 ml toluene was added to the residue. The organic layer extracted with a mixture of water (80 ml) and aqueous HCl (20 ml). The aqueous layer pH was adjusted to about 9.0 with aqueous ammonia and extracted with toluene, (1×100; 2×50 ml). The toluene organic layer was washed with water, saturated aqueous sodium chloride solution, water. The final organic layer was distilled completely to get 10 gm of escitalopram as residue.

The residue was taken into 30 ml of isopropyl alcohol and stirred. 5 gm of oxalic acid was added to the reaction solution and heated to about 50-60° C. The reaction mixture was stirred for 30 minutes, cooled to room temperature and stirred for 30 minutes. The solid was filtered and washed with isopropanol. The wet solid was dried to get 11.5 gm of escitalopram oxalate.

### Example 3

### Purification of Escitalopram Oxalate

30 gm of Escitalopram oxalate (R-isomer content: 1.1%) was added to 60 ml methanol, stirred for about 45 minutes at about 25-40° C. The reaction mixture was filtered on filtration cloth to separate the un-dissolved solid and washed with 5 ml of methanol. 6.5 gm of wet compound (R-isomer content: 1.5%) obtained was dried and kept aside.

The methanol solution obtained as filtrate from the above filtration was heated to about 40° C. and activated charcoal was added. The reaction mixture was filtered on Hyflow® bed and washed with 20 ml methanol. The total methanol solution was distilled off completely under vacuum at 60-65° C. 10 ml of isopropyl alcohol was added and distilled completely. 75 ml of isopropyl alcohol was charged again at 50-60° C., stirred for about 20 minutes. The reaction mixture was cooled to room temperature and stirred for about 45 minutes. Filtered the solid and washed with 10 ml of isopropyl alcohol. The wet solid was dried to get 23 gm of pure escitalopram oxalate. R-isomer content by HPLC: 0.39% w/w

### Example 4

### Preparation of Crystalline Form S of Citalopram Diol

50 gm of 5-Cyano phthalide was stirred in 150 ml of THF at −5° C. 227 gm of 1-(4-fluorophenyl)magnesium bromide was added to the above solution and stirred for about 15 minutes at −5° C. 324 gm of 1-[3-(dimethylamino) propyl]magnesium chloride was added slowly to the reaction mass at −5° C. and then stirred for about 30 minutes at room temperature. The reaction mixture was quenched into cold water (650 ml). Reaction mixture pH was adjusted to about 2 with 36% aqueous HCl (94 ml) and washed the reaction mixture with toluene (2×150 ml). The aqueous layer was separated, toluene (300 ml) was added and pH was adjusted to about 7.5 with aqueous ammonia (45 ml). The organic layer was separated and aqueous layer was extracted with toluene (1×200 ml; 1×100 ml). Total organic layer was washed with water (1×250 ml) followed by salt solution (1×250 ml). The final organic layer was distilled completely under vacuum to get 76 gm of residue.

The residue obtained above was charged in 150 ml of toluene, stirred at room temperature for about 1 hour. The solution was cooled to 10-15° C. and stirred for about 1 hour. The solid was filtered and washed with toluene. The wet solid was dried to get 55 gm of the title compound as crystalline Form S.

The obtained product was analyzed by DSC and XRD, the results are as provided in FIGS. 1 & 2 respectively.

### Example 5

### Preparation of Citalopram

30 gm of crystalline Form S of citalopram diol intermediate obtained in Example 4 was charged in 150 ml of water at room temperature. 30 ml of sulfuric acid was slowly added to the reaction mixture and maintained the reaction mixture at 70° C. for about 6 hours. After completion of the reaction, the reaction mass was quenched into ice cold water (300 ml) and extracted into toluene (350 ml). Total toluene layer was separated and acidified to pH 4.0 with 40 ml of 20% aqueous acetic acid. The aqueous layer was separated and basified to pH 9.0 with aqueous ammonium hydroxide solution. The aqueous layer was extracted with toluene (350 ml). The organic layer was washed with water (200 ml) and saturated aqueous sodium chloride solution (200 ml). The final organic layer was separated and distilled off completely under vacuum to obtain 23 gm of the residue. 46 ml of methanol was added to the residue stirred for dissolution. 23 ml of water was added to the solution and stirred for about 1 hour. Cooled the reaction mixture to 0-5° C., filtered the solid and dried to give 22 gm of the title compound.

### Purity by HPLC: 99.68%

### Example 6

### Preparation of Citalopram Using P-Toluene Sulfonyl Chloride

50 gm of citalopram diol intermediate crystalline Form S obtained in Example 4 was charged in 500 ml of toluene and stirred. 110 ml of ethyl acetate was added to the reaction mass and cooled to about 18° C. 31.5 gm of p-toluene sulfonyl chloride was added to the reaction mixture and maintained for about 2 hours at 25° C. After completion of the reaction, 100 ml of 10% aqueous sodium chloride solution and stirred for 15 minutes. The toluene layer was separated and washed with (2×200 ml) water and (3×150 ml) of aqueous sodium chloride solution. The toluene layer was treated with activated charcoal and distilled off completely to get 40 gm of residue. 80 ml of methanol was added to the residue and stirred for dissolution. 40 ml of water was added slowly to the solution and maintained for 60 minutes. The solid was filtered and dried to get 35 gm of citalopram.

### Purity by HPLC: 99.78%

### Example 7

### Preparation of Citalopram Hydrobromide

About 20 gm of citalopram obtained from the Example 5 was dissolved in 60 ml of Isopropanol at room temperature. The reaction mass was heated to 60-70° C. and pH was adjusted to about 2.0 with 12 ml of 40% hydrogen bromide solution in acetic acid. The reaction mixture was stirred at 25-35° C. for about 1 hour, the suspension was filtered and washed with isopropanol to obtain 21 gm of citalopram hydrobromide.

### Purity by HPLC: 99.64%

### Example 8

### Resolution of citalopram diol crystalline Form S

About 100 gm of citalopram diol crystalline form S obtained form Example 4 was dissolved in 500 ml of isopropanol and stirred to dissolve the crystals. 50 gm of (+)-DPTTA was added to the above solution and stirred for one hour at room temperature to isolation of the solid. The reaction mixture was heated to 45° C. stirred for about 2 hours. The reaction mixture was cooled to room temperature and filtered the solid. The wet compound was added to isopropanol (240 ml), heated to reflux and maintained for 30 minutes. Cooled the reaction mixture to room temperature and stirred for 30 minutes. The solid was filtered and dried to get 54 gm of S-citalopram diol intermediate (+)DPTTA acid salt.

### Example 9

### Preparation of Escitalopram Oxalate

About 24 gm of S-citalopram diol intermediate (+)-DPTTA salt obtained from the Example 8 was stirred in water; pH was adjusted to about 9.0 with aqueous ammonia solution. The reaction mixture was extracted with toluene (1×200 ml, 2×100 ml) and the total organic layer was washed with water. The organic layer was further washed with saturated aqueous sodium chloride solution and final organic layer was distilled off completely to obtain 14 gm of residue.

The residue was charged in 150 ml of toluene and stirred. 29 ml of triethylamine was charged and the reaction mixture was cooled to −5° C. and p-toluene sulfonyl chloride (8.5 ml) was added slowly. The reaction mixture was maintained for about 3 hours. After completion, the reaction mixture was washed with water and aqueous saturated sodium chloride solution (100 ml). The organic layer was distilled completely and 150 ml toluene was added to the residue. The organic layer extracted with a mixture of water (80 ml) and aqueous HCl (20 ml). The aqueous layer pH was adjusted to about 9 with aqueous ammonia and extracted with toluene, (1×100; 2×50 ml). The toluene organic layer was washed with water, saturated aqueous sodium chloride solution, water. The final organic layer was distilled completely to get 12 gm of Escitalopram as residue.

The residue was taken into 36 ml of isopropyl alcohol and stirred. 6 gm of oxalic acid was added to the reaction solution and heated to about 50-60° C. The reaction mixture was stirred for 30 minutes, cooled to room temperature and stirred for 30 minutes. The solid was filtered and washed with isopropanol. The wet solid was dried to get 13 gm of Escitalopram oxalate.

### Purity by HPLC: 99.76%

### Example 10

### Purification of Escitalopram Oxalate

20 gm of escitalopram oxalate (having single maximum impurity of 0.17% w/w) was charged into clean round bottomed flask containing acetone (60 ml) and heated to reflux. The reaction mixture was stirred for 30 minutes and 1 ml of water was added at reflux. The reaction mixture was stirred for another 30 minutes at reflux followed by cooling to 25-35° C. The reaction contents were stirred for 20 minutes and filtered the solid. The solid was dried to get 19 gm of pure escitalopram oxalate having single maximum impurity of 0.06% w/w as determined by HPLC.

1. A crystalline Form S of citalopram diol intermediate of Formula III 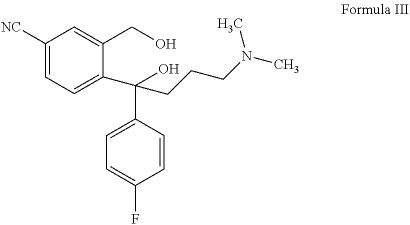
having an endotherm at 77.7±3° C. as measured by differential scanning calorimetry (DSC).

2. The crystalline Form S of citalopram diol intermediate according to claim 1, having an X-ray powder diffraction pattern comprising peaks expressed in degrees 2θ that are selected from 7.36±0.2, 8.43±0.2, 9.77+0.2, 11.79±0.2, 16.86±0.2, 20.08±0.2 and 23.27±0.2.

3. The crystalline Form S of citalopram diol intermediate according to claim 1 having DSC pattern substantially as shown in FIG. 1.

4. A process for the preparation of crystalline Form S of citalopram diol intermediate of claim 1 comprising crystallizing said citalopram diol intermediate from a solvent medium comprising aromatic hydrocarbon solvent.

5. The process of claim 4, wherein said aromatic hydrocarbon solvent is toluene or xylene.

6. The process of claim 4, wherein said citalopram diol solution is prepared by dissolving said citalopram diol intermediate in said solvent medium.

7. The process of claim 4, wherein said citalopram diol intermediate is in the form of oil, amorphous form crystalline form other than Form S.

8. A process for the preparation of citalopram of Formula I 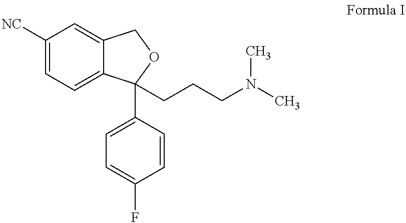
or a salt thereof, comprising converting crystalline Form S of citalopram diol intermediate having Formula III 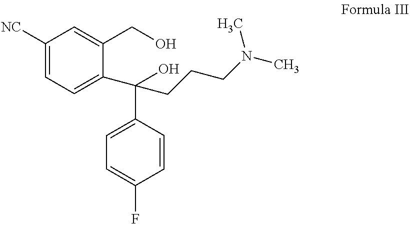
to citalopram.

9. A process for preparation of Escitalopram of Formula II 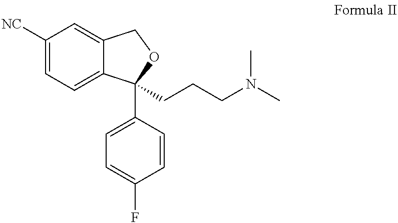
or a salt thereof comprising
a) reacting 5-Cyano phthalide with 1-(4-fluorophenyl)magnesium halide and 1-[3-(dimethylamino) propyl]magnesium halide to obtain citalopram diol of Formula III; 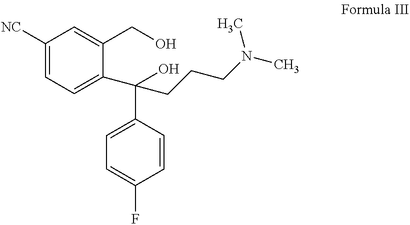
b) reacting citalopram diol of Formula III with an optically pure di-para-toluoyl tartaric acid ((+)DPTTA) to obtain (−)4-(4-(dimethylamino)-1-(4-fluorophenyl)-1-hydroxybutyl)-3-(hydroxymethyl)benzonitrile (+)DPTTA salt of Formula IV in solid form; and 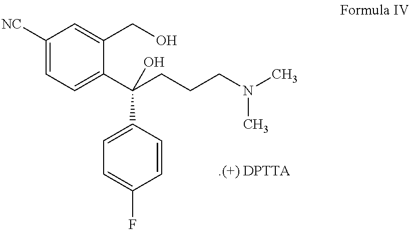
c) converting the (+)DPTTA salt of 4-(4-(dimethylamino)-1-(4-fluorophenyl)-1-hydroxybutyl)-3-(hydroxymethyl)benzonitrile (Formula IV) to Escitalopram of Formula II or a salt thereof.

10. The process of claim 9, wherein said citalopram diol of step b) is in non-crystalline form.

11. The process of claim 9, wherein said citalopram diol of step b) is in residue form.

12. The process of claim 9, wherein said citalopram diol of step b) is crystalline Form S of citalopram diol.

13. The process of claim 9, wherein said step c) involves converting (+)DPTTA salt (−)4-(4-(dimethylamino)-1-(4-fluorophenyl)-1-hydroxybutyl)-3-(hydroxymethyl) benzonitrile of Formula IV into its freebase and reacting said freebase with methane sulfonyl chloride in dichloromethane.

14. A process for purification of Escitalopram oxalate comprising:
a) treating Escitalopram oxalate having more than 1% by weight of R-isomer with an alcohol;
b) removing the un-dissolved solid; and
c) recovering the purified Escitalopram oxalate from the mother liquors.

15. The process of claim 14 wherein the alcohol solvent is selected from methanol, ethanol, n-propanol, isopropanol, n-butanol, isobutanol, and t-butanol.

16. The process of claim 14 wherein said purified Escitalopram oxalate has less than about 1.0% by weight of the R-isomer.

17. A process for purification of Escitalopram oxalate comprising:
a) treating Escitalopram oxalate having more than 1% of single maxiumum impourity content with aqueous acetone;
b) recovering the purified Escitalopram oxalate.

18. The process of claim 17 wherein the aqueous acetone solvent has water content from about 1 to 10% w/w.

19. The process of claim 17 wherein said purified Escitalopram oxalate has a single maximum impurity content less than 0.1%.

20. A process for the preparation of citalopram or a salt thereof comprising reacting citalopram diol intermediate of the Formula III 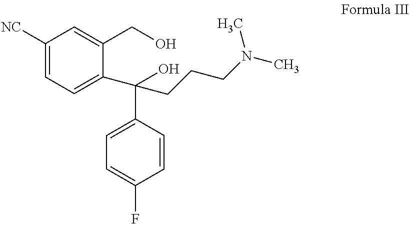
with p-toluenesulfonyl chloride.

21. A process for the preparation of Escitalopram or a salt thereof comprising reacting free base of Formula IV with p-toluenesulfonyl chloride.

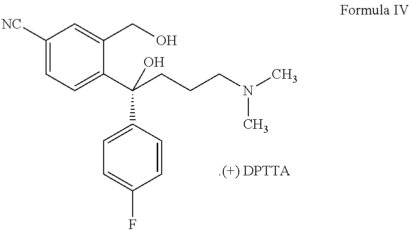

22. The process of claim 4, wherein said citalopram diol intermediate solution is obtained from a preceding process step.

23. The process of claim 17, further comprising heating the mixture of step a) to reflux.

24. The process of claim 23, further comprising cooling said heated mixture.

25. The process of claim 24, wherein said mixture is cooled to about 25° C. to about 35° C.
